(19) United States
(12) Patent Application Publication
(10) Pub. No.: US 2004/0142008 A1
(43) Pub. Date: Jul. 22, 2004

(54) Composition containing fibers and polyurethane, methods

(75) Inventors: Veronique Chevalier, Villecresnes (FR); Annick Collette, St Maur Des Fosses (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/700,503

(22) Filed: Nov. 5, 2003

Related U.S. Application Data
(60) Provisional application No. 60/427,929, filed on Nov. 21, 2002.

(30) Foreign Application Priority Data
Nov. 6, 2002 (FR) 02 13875

(51) Int. Cl. A61K 7/06; A61K 7/11

(52) U.S. Cl. 424/401; 424/70.17

(57) ABSTRACT

The invention relates to a composition containing fibers and an associative polyurethane useful for example as a care or makeup composition for keratin materials.

### REFERENCE TO PRIOR APPLICATIONS

This application claims priority to U.S. provisional application 60/427,929 filed Nov. 21, 2002, and to French patent application 0213875 filed November 6, 2002, both incorporated herein by reference.

### FIELD OF THE INVENTION

The present invention relates to a composition preferably in a form suitable for topical application such as in emulsion form, containing fibers and at least one associative polyurethane, and to its uses especially in cosmetics and dermatology, in particular to care for, treat or make up keratin materials such as the skin, including the scalp, the lips of the face and the integuments such as the eyelashes, the eyebrows, the nails and the hair. The present invention also relates to the use of an associative polyurethane, in a composition containing fibers, to obtain a composition that is smooth and uniform, even after a certain period of use.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known practice to incorporate fibers into cosmetic compositions. Thus, document JP 07-196 440 describes cosmetic compositions containing short polyamide fibers, these fibers giving the said compositions a velvety feel and good cosmetic behaviour. Moreover, in the field of skin makeup, it is known practice to use fibers in makeup products, especially for their lengthening effects in mascaras (see JP-A-57/158 714), their “textile” feel (see JP-A-7/196 440), their fabric effect or their moisturizing properties in lipsticks (see document U.S. Pat No. 5,498,407) or to improve the contours of lipstick on the edges of the lips (see document EP-A-0 106 762).

These fibers may especially be incorporated into either water-in-oil (W/O) emulsions or oil-in-water (O/W) emulsions, as described, for example, in documents EP-A-1 090 626, EP-A-1 090 627, EP-A-1 092 424 and EP-A-1 092 425.

Unfortunately, over time, especially when the fibers are in high proportion, for example in a proportion of greater than 5% by weight, and especially when the compositions are in the form of emulsions, the compositions containing fibers develop a stringy appearance during use, and consequently become less pleasant to see and use.

There is thus still a need for a composition for topical application, especially in emulsion form, which contains fibers and which does not have the above drawbacks, i.e. which remains smooth and uniform throughout the time of use by the consumer.

### DETAILED DESCRIPTION OF THE PREFERRED EMBODIMENTS

The inventors have found, surprisingly, that by using an associative polyurethane, a composition with very good stability and good cosmetic properties can be obtained.

The invention applies not only to care, treatment and/or makeup products for human skin, both of the face and of the body, including the scalp, and the lips of the face, but also for example to makeup products for the integuments, for instance the eyelashes, the eyebrows and the nails, and to haircare and/or hair treatment products.

More specifically, one subject of the invention is a composition for topical application comprising, in a physiologically acceptable medium, fibers and at least one associative polyurethane, the medium comprising an aqueous phase and an oily phase comprising at least one oil.

The expression “topical application” means herein an external application to keratin materials, which are especially the skin, the scalp, the eyelashes, the eyebrows, the nails, mucous membranes and the hair.

The expression “physiologically acceptable medium” means a medium that is compatible with the skin, the lips, the nails, the scalp and/or the hair.

The composition of the invention is pleasantly soft when applied to the keratin materials and especially to the skin, and has good cosmetic behaviour. In addition, by virtue of the associative polyurethane, a composition that remains uniform and stable over time and that does not acquire a stringy appearance is obtained. In addition, this composition has the advantage of being “self-smoothing”, i.e. after each use, a smooth and uniform surface reforms at the surface of the jar, as if the jar was new, and when the cream is taken up on a finger or any other means (sponge or spatula), the mark of this finger or of this means does not remain at all.

Thus, further subjects of the invention are the use of an associative polyurethane in a composition for topical application, containing fibers, to obtain a composition that is constantly smooth and uniform, and the invention compositions contained in, located in, or present in, (hereinafter “contained in”) a container means, such as for example a jar, can, bottle, or the like.

The expression “constantly smooth and uniform” means a composition that does not acquire a stringy appearance over time and whose surface regains its smoothness after use.

The composition of the invention generally preferably has the appearance of a cream, i.e. it generally may have a viscosity ranging from 10 to 300 poises (i.e. 1 to 30 Pa.s), preferably from 30 to 250 poises (i.e. 3 to 25 Pa.s) and better still from 75 to 250 poises (i.e. 7.5 to 25 Pa.s), measured at 25° C. using a Rheomat 180 viscometer.

Fibers

In the present patent application, the term “fiber” should be understood as meaning an object of length L and of diameter D such that L is very much greater than D, D being the diameter of the circle in which the cross section of the fiber is inscribed. In particular, the ratio L/D (or shape factor) is preferably chosen in the range from 3.5 to 2 500, more preferably from 5 to 500 and better still from 5 to 150.

The fibers may be present in the composition according to the invention in any amount, including for example an amount ranging from 0.5% to 50% by weight, preferably from 5% to 40% by weight, better still from 5% to 30% by weight and even better still from 10% to 20% by weight relative to the total weight of the composition.

The fibers that may be used in the composition of the invention include fibers of synthetic or natural, mineral or organic origin. They may be short or long, and individual or organized, for example braided. They may have any form and may especially have a circular or polygonal (square, hexagonal or octagonal) cross section depending on the intended specific application. In particular, their ends are blunted and/or polished to prevent injury.

In a preferred embodiment, the fibers range from 1 nm to 20 mm, more preferably from 10 nm to 5 mm and better still from 0.1 mm to 1.5 mm in length, and their cross section may be within a circle of diameter ranging from 2 nm to 100 μm, more preferably ranging from 20 nm to 20 μm and better still from 5 μm to 50 μm. Moreover, they are characterized by their weight, often given in denier or decitex.

The fibers may be those used in the manufacture of textiles and especially silk fiber, cotton fiber, wool fiber, flax fiber, cellulose fiber extracted especially from wood, from vegetables or from algae, polyamide (Nylon®, especially under the names Nylon 6=Polyamide 6; Nylon 6,6=Polyamide 66) fiber; rayon fiber, viscose fiber, acetate fiber, especially rayon acetate, cellulose acetate or silk acetate fiber, poly-p-phenyleneterephtalamide fiber, especially Kevlar® fiber, acrylic fiber, especially polymethyl methacrylate fiber, or poly(2-hydroxyethyl methacrylate) fiber, polyolefin fiber and especially polyethylene or polypropylene fiber, glass fiber, silica fiber, aramid fiber, carbon fiber, especially in graphite form, Teflon® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, fibers formed from a mixture of polymers such as those mentioned above, for instance polyamide, polyester fibers, and surgical fibers, and mixtures of these fibers.

Surgical fibers that may be used include resorbable synthetic fibers prepared from glycolic acid and caprolactone (“Monocryl” from the company Johnson & Johnson); resorbable synthetic fibers of the lactic acid/glycolic acid copolymer type (“Vicryl” from the company Johnson & Johnson); terephthalic polyester fibers (“Ethibond” from the company Johnson & Johnson) and stainless-steel yarns (“Steel” from the company Johnson & Johnson).

Moreover, the fibers may or may not be surface-treated, and may or may not be coated. As coated fibers that may be used in the invention, mention may be made of polyamide fibers coated with copper sulphide for an antistatic effect (for example the R-STAT fibers from the company Rhodia) or another polymer allowing a particular organization of the fibers (specific surface treatment) or surface treatment inducing colour/hologram effects (“Lurex” fiber from the company Sildorex, for example).

The fibers that may be used in the composition according to the invention are preferably flexible fibers. The composition is preferably free of rigid fibers.

According to one preferred embodiment of the invention, the fibers that may be used in the composition according to the invention are polyamide fibers and poly-p-phenyleneterephtalamide fibers. They may preferably range from 0.1 to 5 mm and more preferably from 0.25 to 1.6 mm in length and their mean diameter may range from 5 to 50 μm.

According to one preferred embodiment of the invention, the fibers are chosen from Nylon 6 (or Polyamide 6) (INCI name: Nylon 6) fiber and Nylon 6,6 (or Polyamide 66) (INCI name: Nylon 66) fiber, and mixtures thereof.

In particular, it is possible to use the polyamide fibers sold by Etablissements P. Bonte under the name Polyamide 0.9 Dtex 0.3 mm (INCI name: Nylon 66), having a mean diameter of 6 μm, a weight of about 0.9 dtex and a length ranging from 0.3 mm to 1.5 mm. It is also possible to use poly-p-phenylene terephtalamide fibers with a mean diameter of 12 μm and a length of about 1.5 mm, for instance those sold under the name Kevlar Floc by the company Du Pont Fibers.

Associative Polyurethane

Associative polyurethanes are nonionic block copolymers comprising in the chain both hydrophilic blocks usually of polyoxyethylenated nature and hydrophobic (lipophilic) blocks that may be aliphatic sequences alone and/or cycloaliphatic and/or aromatic sequences.

In particular, these copolymers comprise at least two hydrocarbon-based lipophilic chains containing from 6 to 30 carbon atoms (C₆ to C₃₀), separated by a hydrophilic block, the hydrocarbon-based chains possibly being pendent chains or chains at the end of a hydrophilic block. In particular, it is possible for one or more pendent chains to be provided. In addition, the polymer may comprise a hydrocarbon-based chain at one or both ends of a hydrophilic block.

The polymers may be arranged in blocks in triblock or multiblock form. The hydrophobic blocks may thus be at each end of the chain (for example: triblock copolymer with a hydrophilic central block) or distributed both at the ends and in the chain (for example multiblock copolymer). The polymers may also be graft or starburst polymers.

The hydrophilic block is preferably an oxyalkylenated chain, in particular an oxyethylene chain.

Thus, the polymers preferably contain an oxyethylenated hydrophilic chain. In addition, they are preferably triblock polymers, especially triblock polymers in which the hydrophilic block is an oxyethylenated chain, comprising from 50 to 1 000 oxyethylene groups and preferably from 70 to 500 oxyethylene groups.

In general, associative polyurethanes comprise a urethane bond between the hydrophilic blocks, hence their name. By extension, also included among associative polyurethanes are polymers in which the hydrophilic blocks are linked via other chemical bonds to lipophilic blocks. However, according to one preferred embodiment of the invention, the associative polyurethane used contains a urethane bond. These polyurethanes may be used in pure form or dissolved or dispersed in water or in aqueous-alcoholic media.

As examples of associative polymers that may be used in the invention, mention may be made of the polymer C₁₆-OE₁₃₆-C₁₆, OE being an oxyethylene unit, sold by the company Hüls under the name SER-AD FX1100, which is a molecule containing a urethane function, with a weight average molecular weight of 30 000 (INCI name: Steareth-100/PEG-136/HMDI copolymer). Associative polymers that may also be used include polyurethanes containing polyether and urea groups, sold under the names Rheolate 205, Rheolate 204 and Rheolate 208 by the company Rheox (INCI name: Polyether-urea-polyurethane). These associative polyurethanes are sold in pure form.

Examples of solutions or dispersions of these polymers that may be mentioned include the polyurethanes sold under the names SER-AD FX1010 and SER-AD 1035 by the company Hüls (INCI name: Polyurethane), those sold under the names Rheolate 255, Rheolate 278 and Rheolate 244 by the company Rheox (INCI name: Polyether-urea-polyurethane), those sold under the names DW 1206F, DW 1206J, DW 1206B and DW 1206G by the company Röhm & Haas (INCI name: Polyurethane) and the product sold under the name Acrysol RM 2020 by the company Röhm & Haas.

The polymers that may be used in the invention include in particular those described in the article by G. Fonnum, J. Bakke and Fk. Hansen—Colloid Polym. Sci 271, 380-389 (1993).

According to one preferred embodiment of the invention, a water-soluble polyurethane is used, since a polyurethane solution is more suitable for the purpose of the invention than a dispersion.

The composition according to the invention contains one or more associative polyurethanes, in an amount that is sufficient to obtain the desired aim, i.e. to obtain a composition that remains smooth, that does not acquire a stringy appearance over the time of use by the consumer, and that is self-smoothing.

The amount of associative polyurethane(s) as active material is not limited and may range, for example, from 0.5% to 10% by weight, preferably from 1% to 5% by weight and better still from 1.5% to 3% by weight relative to the total weight of the composition.

The composition according to the invention comprises an oily phase and an aqueous phase. It may contain an oily continuous or outer phase (W/O) or an aqueous continuous phase (O/W), and it may be a simple emulsion (O/W or W/O emulsion) or a triple emulsion (W/O/W or O/W/O emulsion) or a multiple emulsion. It is generally, in particular, in the form of an O/W emulsion constituting a cream or an ointment.

When the emulsion is of the oil-in-water type, the associative polyurethane makes it possible to obtain a polymer “network” on the skin, during the application, especially enabling self-cicatrization and levelling-out of the relief of the skin.

According to one preferred embodiment of the invention, the continuous phase is an aqueous phase and the composition is in the form of O/W emulsions. Compositions containing an aqueous continuous or outer phase have the advantage of spreading easily, of being light, of penetrating into the skin well, of not being sticky and of providing freshness to the skin when applied, unlike compositions containing an oily continuous phase.

Aqueous Phase

Advantageously, the aqueous phase contains water and optionally one or more water-miscible or at least partially water-miscible compounds, for instance polyols; C₂ to C₈ lower monoalcohols, such as ethanol and isopropanol; and C₃ to C₄ ketones that are liquid at room temperature. The term “room temperature” should be understood as meaning a temperature of about 25° C., at normal atmospheric pressure (760 mmHg).

The term “polyol” should be understood as meaning any organic molecule comprising at least two free hydroxyl groups. Examples of polyols that may be mentioned include glycerol, glycols, for instance butylene glycol, propylene glycol, and isoprene glycol, and polyethylene glycols, for instance PEG-8, sorbitol and sugars, for instance glucose.

The aqueous phase may also comprise any common water-soluble or water-dispersible additive as mentioned below.

The amount of aqueous phase is not limited and may preferably represent from 10% to 95% by weight, preferably from 20% to 90% by weight, better still from 30% to 80% by weight and even better still from 40% to 70% by weight relative to the total weight of the composition.

Any water-miscible compound(s), such as lower polyols and alcohols, similarly are not limited, and preferably may be present in an amount ranging from 0 to 30%, especially from 0.1% to 30% and better still in an amount ranging from 1% to 20%, relative to the total weight of the composition.

Oily Phase

The oily phase is a fatty phase that is liquid at room temperature (25° C.). It contains at least one fatty substance chosen from volatile or non-volatile oils that are liquid at room temperature (20-25° C.), gums and pasty fatty substances of animal, plant, mineral or synthetic origin, and mixtures thereof. These fatty substances are physiologically acceptable.

The oily phase may also comprise any common liposoluble or lipodispersible additive as mentioned below.

The oily phase contains at least one oil. The term “oil” means a fatty substance that is liquid at room temperature (25° C.).

As oils that may be used in the composition of the invention, examples that may be mentioned include:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;

synthetic esters and ethers, especially of fatty acids, for instance the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol didctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, isohexadecane, isododecane, and hydrogenated polyisobutene such as Parleam® oil;

fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol;

partially hydrocarbon-based and/or silicone-based fluoro oils, for instance those described in document JP-A-2 295 912;

silicone oils, for instance volatile or non-volatile polymethylsiloxanes (PDMSs) containing a linear or cyclic silicone chain, which are liquid or pasty at room temperature, especially cyclopolydimethylsiloxanes (cyclomethicones) such as cyclopentasiloxane and cyclo-hexadimethylsiloxane; polydimethylsiloxanes comprising alkyl, alkoxy or phenyl groups, which are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyl-dimethicones, diphenylmethyldiphenyltrisiloxanes, 2-phenylethyltrimethylsiloxysilicates and polymethyl-phenylsiloxanes;

mixtures thereof.

According to one preferred embodiment, the composition of the invention comprises at least one oil chosen from silicone oils and fatty acid esters, and mixtures thereof. According to a more particularly preferred embodiment, the composition of the invention comprises at least one volatile silicone oil, especially a cyclopolydimethylsiloxane.

The other fatty substances that may be present in the oily phase are, for example, fatty acids containing from 8 to 30 carbon atoms, for instance stearic acid, lauric acid or palmitic acid; gums such as silicone gums (dimethiconol); silicone resins such as trifluoromethyl-C₁₋₄ alkyldimethicone and trifluoropropyldimethicone, and silicone elastomers, for instance the products sold under the names “KSG” by the company Shin-Etsu, under the name “Trefil” by the company Dow Corning or under the name “Gransil” by the company Grant Industries, and mixtures thereof.

These fatty substances may be chosen in view of this disclosure in a varied manner by a person skilled in the art in order to prepare a composition having the desired properties, for example in terms of consistency or texture.

According to one preferred embodiment of the invention, the composition is wax-free.

The amount of oily phase in the composition of the invention is not limited and preferably may range from 5% to 90% by weight, more preferably from 10% to 80% by weight, better still from 20% to 70% by weight and even better still from 30% to 60% by weight relative to the total weight of the composition. The amount of oil(s) is preferably at least 5% by weight relative to the total weight of the composition and preferentially at least 8% by weight relative to the total weight of the composition.

Emulsifier

The emulsions of the invention generally can contain at least one emulsifier chosen from amphoteric, anionic, cationic and nonionic emulsifiers, used alone or as a mixture, and optionally a co-emulsifier. The emulsifiers are chosen in a suitable manner according to the emulsion to be obtained (W/O or O/W emulsion). The emulsifier(s) is (are) preferably chosen from nonionic emulsifiers.

The emulsifier and the co-emulsifier are generally present in the composition in an active-material proportion that can range, for example, from 0.05% to 30% by weight, preferably from 0.2% to 20% by weight and better still from 0.5% to 10% by weight, relative to the total weight of the composition.

Examples of emulsifiers that may be mentioned for the W/O emulsions include dimethicone copolyols such as the mixture of cyclomethicone and of dimethicone copolyol, sold under the name “DC 5225 C” by the company Dow Corning, and alkyldimethicone copolyols, such as the laurylmethicone copolyol sold under the name “Dow Corning 5200 Formulation Aid” by the company Dow Corning, the cetyldimethicone copolyol sold under the name Abil EM 90® by the company Goldschmidt, or the mixture of cetyldimethicone copolyol, polyglyceryl-4 isostearate and hexyl laurate, sold under the name Abil WE 09 by the company Goldschmidt. One or more co-emulsifiers may also be added thereto, which may be advantageously chosen from the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Emulsifiers for W/O emulsions that may also be used include a crosslinked elastomeric solid organopolysiloxane comprising at least one oxyalkylenated group, such as those obtained according to the procedure of Examples 3, 4 and 8 of document U.S. Pat No. 5,412,004 and the examples of document U.S. Pat No. 5,811,487, especially the product of Example 3 (synthesis example) of patent U.S. Pat No. 5,412,004, and such as the product sold under the reference KSG 21 by the company Shin Etsu.

Examples of emulsifiers that may be mentioned for the O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; the ethers of fatty alcohols; the sugar ethers of C₈-C₂₄ fatty alcohols, and mixtures thereof.

An oxyalkylenated fatty acid ester of sorbitol that may especially be mentioned is the ester of sorbitan and of isostearic acid containing 20 oxyethylene groups (INCI name: PEG-20 Sorbitan Isostearate), such as the product sold under the name Nikkol TI 10 V by the company Nikkol.

Glyceryl esters of fatty acids that may especially be mentioned include glyceryl stearate (glyceryl mono-, di- and/or tristearate)(INCI name: glyceryl stearate) or glyceryl ricinoleate and mixtures thereof.

Polyethylene glycol esters of fatty acids that may especially be mentioned include polyethylene glycol stearate (polyethylene glycol mono-, di- and/or tristearate) and more especially polyethylene glycol 50 OE monostearate (INCI name: PEG-50 stearate) and polyethylene glycol 100 OE monostearate (INCI name: PEG-100 stearate), and mixtures thereof.

It is also possible to use mixtures of these surfactants, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (INCI name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (INCI name: Methyl glucose dioleate); the mixed ester of methylglucoside and of the oleic acid/hydroxystearic acid mixture (INCI name: Methyl glucose dioleate/hydroxystearate); the ester of methylglucoside and of isostearic acid (INCI name: Methyl glucose isostearate); the ester of methylglucoside and of lauric acid (INCI name: Methyl glucose laurate); the mixture of the monoester and diester of methylglucoside and of isostearic acid (INCI name: Methyl glucose sesquiisostearate); the mixture of the monoester and diester of methylglucoside and of stearic acid (INCI name: Methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (INCI name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (INCI name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate, and mixtures thereof.

Examples of fatty alkyl ethers that may be mentioned include ethers of polyethylene glycol and of fatty alcohol containing from 8 to 30 carbon atoms and especially from 10 to 22 carbon atoms, such as the polyethylene glycol ethers of cetyl, stearyl or cetearyl alcohol (mixture of cetyl alcohol and stearyl alcohol). Mention may be made, for example, of ethers containing from 1 to 200 and preferably from 2 to 100 oxyethylenated groups, such as those of INCI name Ceteareth-20 and Ceteareth-30, and mixtures thereof.

Sugar ethers that may especially be mentioned are alkylpolyglucosides, for example decylglucoside, for instance the product sold under the name Mydol 10 by the company Kao Chemicals, the product sold under the name Plantaren 2000 by the company Henkel, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Henkel, and also arachidyl glucoside, for example in the form of the mixture of arachidyl alcohol and behenyl alcohol and arachidyl glucoside, sold under the name Montanov 202 by the company SEPPIC, and mixtures thereof.

According to one preferred embodiment of the invention, the composition is in the form of an O/W emulsion, and the emulsifier is chosen from oxyalkylenated fatty acid esters of sorbitol, and in particular the emulsifier is the ester of sorbitan and of isostearic acid containing 20 oxyethylene groups; these emulsifiers make it possible to obtain a composition that is both smooth, self-healing and very stable, and also having very good cosmetic properties.

Additives

The composition of the invention may also contain one or more adjuvants, for example those that are common in cosmetics or dermatology. Adjuvants that may be mentioned include gelling agents, active agents, preserving agents, antioxidants, fragrances, solvents, fillers, sunscreens (=UV-screening agents), dyestuffs, basic agents (triethanolamine, diethanolamine or sodium hydroxide) or acidic agents (citric acid), and also lipid vesicles or any other type of vector (nanocapsules, microcapsules, etc.), and mixtures thereof. These adjuvants are used in the usual proportions in the cosmetics field, for example from 0.01% to 30% of the total weight of the composition, and, depending on their nature, they are introduced into the aqueous phase of the composition or into the oily phase, or alternatively into vesicles or any other type of vector. These adjuvants and the concentrations thereof should be such that they do not modify the desired property for the emulsion of the invention.

Thus, depending on the fluidity of the composition that it is desired to obtain, it is possible to incorporate into the composition one or more gelling agents, especially hydrophilic gelling agents, i.e. agents that are soluble or dispersible in water. Examples of hydrophilic gelling agents that may be mentioned include modified or unmodified carboxyvinyl polymers, such as the products sold under the names Carbopol (INCI name: carbomer) and Pemulen (INCI name: Acrylates/C10-30 alkyl acrylate cross polymer) by the company Goodrich; polyacrylamides; optionally crosslinked and/or neutralized 2-acrylamido-2-methylpropane sulphonic acid polymers and copolymers, for instance the poly(2-acrylamido-2-methylpropane-sulphonic acid) sold by the company Hoechst under the name “Hostacerin AMPS” (INCI name: ammonium polyacryldimethyltauramide); crosslinked anionic copolymers of acrylamide and of AMPS, which are in the form of a W/O emulsion, such as those sold under the name Sepigel 305 (INCI name: Polyacrylamide/C13-14 Isoparaffin/Laureth-7) and under the name Simulgel 600 (INCI name: Acrylamide/Sodium acryloyldimethyltaurate copolymer/Isohexadecane/Polysorbate 80) by the company SEPPIC; polysaccharide biopolymers, for instance xanthan gum, guar gum, alginates and modified or unmodified celluloses; and mixtures thereof.

As fillers that may be used in the composition of the invention, examples that may be mentioned include the pigments such as titanium oxide, zinc oxide or iron oxide and organic pigments; kaolin; silica; talc; boron nitride; organic spherical powders, and mixtures thereof. Examples of organic spherical powders that may be mentioned include polyamide powders and especially Nylon® powders such as Nylon-1 or Polyamide 12, sold under the name Orgasol by the company Atochem; polyethylene powders; Teflon®; microspheres based on acrylic copolymers, such as those made of ethylene glycol dimethacrylate/lauryl methacrylate copolymer, sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; powders of natural organic materials such as starch powders, especially of maize starch, wheat starch or rice starch, which may or may not be crosslinked, such as the starch powders crosslinked with octenyl succinate anhydride, sold under the name Dry-Flo by the company National Starch; and mixtures thereof. These fillers may be present in amounts ranging from 0 to 20% by weight and preferably from 1% to 10% by weight relative to the total weight of the composition.

As active agents that may be used in the composition of the invention, examples that may be mentioned include enzymes (for example lactoperoxidase, lipase, protease, phospholipase and cellulases); flavonoids; moisturizers such as protein hydrolysates; sodium hyaluronate; polyols, for instance glycerol, glycols, for instance polyethylene glycols, and sugar derivatives; antiinflammatories; procyannidol oligomers; vitamins, for instance vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine; depigmenting agents such as kojic acid, hydroquinone and caffeic acid; salicylic acid and its derivatives; α-hydroxy acids such as lactic acid and glycolic acid and derivatives thereof; retinoids such as carotenoids and vitamin A derivatives; hydrocortisone; melatonin; algal extracts, fungal extracts, plant extracts, yeast extracts or bacterial extracts; steroids; antibacterial active agents, for instance 2,4,4′-trichloro-2′-hydroxy-diphenyl ether (or triclosan), 3,4,4′-trichloro-carbanilide (or triclocarban) and the acids indicated above, and especially salicylic acid and its derivatives; tensioning agents; ceramides; essential oils; and mixtures thereof; and any active agent that is suitable for the final aim of the composition.

Examples of steroids that may be mentioned include dehydroepiandrosterone (or DHEA), and also (1) its precursors and biological derivatives, in particular the salts and esters of DHEA, such as DHEA sulphate and salicylate, 7-hydroxy DHEA, 7-keto DHEA, 7-hydroxy and 7-keto DHEA esters, especially 3-β-acetoxy-7-oxo DHEA, and (2) its precursors and chemical derivatives, in particular sapogenins such as diosgenin or hecogenin, and/or derivatives thereof such as hecogenin acetate, and/or natural extracts containing them and especially extracts of Dioscorea plants, such as wild yam.

The UV-screening agents may be organic or mineral (or physical UV sunblocks).

The UV-screening agents may be present in an active-material amount ranging from 0.01% to 20% by weight of active material, preferably from 0.1% to 15% by weight and better still 0.2% to 10% by weight relative to the total weight of the composition.

As examples of UV-A-active and/or UV-B-active organic screening agents that may be added to the composition of the invention, examples that may be mentioned include derivatives containing a sulphonic function, such as sulphone-containing or sulphonate-containing derivatives of benzylidenecamphor, of benzophenone or of phenylbenzimidazole, more particularly benzylidenecamphor derivatives, for instance 1,4-bis(3-methylidenecamphor-10-sulphonic acid) (INCI name: Terephthalylidenedicamphorsulphonic acid) manufactured under the name “Mexoryl SX” by the company Chimex, 3-benzylidenecamphor-4′-sulphonic acid (INCI name: Benzylidenecamphorsulphonic acid), manufactured under the name “Mexoryl SL” by the company Chimex, 2-[4-(camphormethylidene)phenyl]benzimidazole-5-sulphonic acid and phenylbenzimidazolesulphonic acid (INCI name: Phenylbenzimidazolesulphonic acid), sold under the name Eusolex 232 by the company Merck; para-aminobenzoic acid derivatives; salicylic derivatives such as ethylhexyl salicylate sold under the trade name Neo Heliopan OS by Haarmann & Reimer; dibenzoylmethane derivatives such as butyl methoxydibenzoyldimethane sold especially under the trade name Parsol 1789 by Hoffmann La Roche; cinnamic derivatives such as ethylhexyl methoxycinnamate sold especially under the trade name Parsol MCX by Hoffmann La Roche; β,β′-diphenylacrylate derivatives such as octocrylene (2-ethylhexyl α-cyano-β,β-diphenylacrylate) sold under the trade name Uvinul N539 by the company BASF; benzophenone derivatives such as Benzophenone-1 sold under the trade name Uvinul 400 by BASF, Benzophenone-2 sold under the trade name Uvinul D50 by BASF, Benzophenone-3 or Oxybenzone, sold under the trade name Uvinul M40 by BASF, Benzophenone-4 sold under the trade name Uvinul MS40 by BASF; benzylidenecamphor derivatives such as 4-methylbenzylidenecamphor sold under the trade name Eusolex 6300 by Merck; phenyl-benzimidazole derivatives such as Benzimidazilate sold under the trade name Neo Heliopan AP by Haarmann & Reimer; triazine derivatives such as Anisotriazine sold under the trade name Tinosorb S by Ciba Geigy and ethylhexyltriazone sold especially under the trade name Uvinul T150 by BASF; phenylbenzotriazole derivatives such as Drometrizole Trisiloxane sold under the trade name Silatrizole by Rhodia Chimie; anthranilic derivatives such as menthyl anthranilate sold under the trade name Neo Heliopan MA by Haarmann & Reimer; imidazoline derivatives; benzalmalonate derivatives; and mixtures thereof.

As physical sunblocks that may be added to the composition of the invention, examples that may be mentioned include pigments and nanopigments of coated or uncoated metal oxides, especially titanium oxide, iron oxide, zirconium oxide, zinc oxide or cerium oxide, and mixtures thereof, these oxides possibly being in the form of optionally coated microparticles or nanoparticles (nanopigments).

The composition of the invention is preferably intended for topical application and may especially constitute a dermatological or cosmetic composition intended, for example, to care for, treat, cleanse and make up keratin materials and especially human skin, lips, hair, eyelashes and nails.

Thus, it may constitute a treatment or care composition for the skin (including the scalp), for keratin fibers (hair, eyelashes or eyebrows), for the nails or for the lips, or an antisun composition or artificial tanning composition, or alternatively a cleansing or makeup-removing product for the skin, the hair, the eyebrows or the eyelashes, a deodorant product or a fragrancing product. In this case, it is generally uncoloured or weakly coloured, and it may optionally contain cosmetic or dermatological active agents. It may then be used as a care base for the skin or the lips (lip balms, for protecting the lips against the cold and/or sunlight and/or the wind) or as a day or night care cream for facial and/or body skin.

The composition according to the invention may also constitute a coloured cosmetic composition and especially a skin makeup product, in particular a foundation, a blusher, a makeup rouge, an eyeshadow, a mascara or an eyeliner, optionally having care or treatment properties, or a body tattoo.

According to one preferred embodiment of the invention, the composition is intended for topical application to the skin.

Thus, a subject of the invention is also the cosmetic use of a cosmetic composition as defined above to care for, treat, cleanse and/or make up keratin materials, preferably the skin.

A subject of the invention is also a cosmetic care or treatment process for keratin materials and especially human skin, hair or lips, comprising the application to these keratin materials of a composition, in particular a cosmetic composition, as defined above. The keratin material is preferably the skin.

The composition according to the invention is prepared according to the usual methods in the fields of application under consideration.

The invention is illustrated in greater detail in the non-limiting examples that follow. The compounds are indicated, depending on the case, as the INCI (=CTFA) name or as the chemical name, and the percentages are given on a weight basis, unless otherwise mentioned.

### EXAMPLE 1

### White Cream (O/W Emulsion)

| Phase A | |
|---|---|
| Cyclohexasiloxane | 23% |
| Cetyl alcohol | 0.5% |
| Stearyl alcohol | 0.5% |
| PEG-20 sorbitan isostearate (Nikkol TI V 10) | 1.25% |
| Phase B | |
| Glycerol | 5% |
| Preserving agents | 1% |
| Water | qs 100% |
| Phase C | |
| Steareth-100/PEG-136/HMDI copolymer | 2.24% |
| (SER-AD FX1100) | |
| Water | 20.16% |
| Phase D | |
| Polyamide (Nylon 66) fibers | 6% |
| (Polyamide 0.9 dtex, 0.3 mm - Société | |
| Paul Bonte) | |
| Aluminium Starch Octenylsuccinate (Dry Flo) | 3% |

Procedure: The constituents of phase A and phase B are mixed separately with stirring under hot conditions (about 70 to 80° C.) and the oily phase (phase A) is introduced into the aqueous phase (phase B) with stirring. The mixture is cooled to about 40° C., and phase C, prepared beforehand, is then introduced therein with stirring. Finally, phase D is incorporated.

A white cream is obtained, in which the fibers are uniformly distributed and which is very gentle when applied. This cream may be used, for example, for skin care. The deposit obtained is very uniform and remains uniform over time, and no stringy appearance is observed in the composition over the duration of use.

### EXAMPLE 2

### White Cream (O/W Emulsion)

| Phase A | |
|---|---|
| Cyclohexasiloxane | 15% |
| Methyl glucose sesquistearate | 2% |
| Caprylic/capric triglyceride | 10% |
| (Miglyol 812) | |
| Phase B | |
| Glycerol | 7% |
| Preserving agents | .1% |
| Water | qs 100% |
| Phase C | |
| PEG-20 methyl glucose sesquistearate | 3% |
| Water | 19% |
| Phase D | |
| Polyamide (Nylon 66) fibers | 8% |
| (Polyamide 0.9 dtex, 0.3 mm-Société | |
| Paul Bonte) | |
| Phase E | |
| Steareth-100/PEG-136/HMDI copolymer | 2.5% |
| (SER-AD FX1100) | |
| Water | 20.16% |
| Phase F | |
| Kaolin | 2% |

Procedure: The constituents of phase A and phase B are mixed separately with stirring under hot conditions (about 70 to 80° C.), and the oily phase (phase A) is introduced into the aqueous phase (phase B), with stirring. The mixture is cooled to about 40° C., and phase C, prepared beforehand, is then introduced therein with stirring. Finally, phases D, E and F are incorporated.

A white cream is obtained, in which the fibers are uniformly distributed and which is very gentle when applied. This cream may be used, for example, for skin care. The deposit obtained is very uniform and remains uniform over time, and no stringy appearance is observed in the composition over the duration of use.

The above written description of the invention provides a manner and process of making and using it such that any person skilled in this art is enabled to make and use the same, this enablement being provided in particular for the subject matter of the appended claims, which make up a part of the original description and including a composition for topical application, comprising, in a physiologically acceptable medium, fibers and at least one associative polyurethane, the medium comprising an aqueous phase and an oily phase comprising at least one oil. Preferred embodiments of the invention similarly fully described and enabled include: the cosmetic use of a cosmetic composition according to the invention, to care for, treat, cleanse and/or make up keratin materials, preferably the skin; a cosmetic care or treatment process for human keratin materials, comprising the application to these keratin materials of a cosmetic composition according to the invention; and the cosmetic use of an associative polyurethane, in a composition for topical application, containing fibers, to obtain a composition that is constantly smooth and uniform.

As used above, the phrases “chosen from” and “selected from the group consisting of” and the like include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition for topical application, comprising, in a physiologically acceptable medium, fibers and at least one associative polyurethane, the medium comprising an aqueous phase and an oily phase comprising at least one oil.

2. The composition according to claim 1, wherein the fibers are selected from the group consisting of silk fiber, cotton fiber, wool fiber, flax fiber, cellulose fiber, polyamide fiber, rayon fiber, viscose fiber, acetate fiber, poly-p-phenylene-terephtalamide fiber, acrylic fiber, polyolefin fiber, glass fiber, silica fiber, aramid fiber, carbon fiber, especially in graphite form, Teflon® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, fibers of polymer mixtures, surgical fibers, and mixtures thereof.

3. The composition according to claim 1, wherein the fibers are fibers of synthetic origin.

4. The composition according to claim 1, wherein the fibers are polyamide fibers or poly-p-phenyleneterephtalamide fibers.

5. The composition according to claim 1, wherein the fibers are 0.1-1.5 mm in length.

6. The composition according to claim 1, wherein the fibers have a mean diameter of 5-50 μm.

7. The composition according to claim 1, wherein the fibers are selected from the group consisting of Nylon 6 fiber, Nylon 6,6 fiber, and mixtures thereof.

8. The composition according to claim 1, wherein the fibers are present in an amount of 0.5%-50% by weight relative to the total weight of the composition.

9. The composition according to claim 1, wherein the amount of associative polyurethane is 0.5%-10% by weight relative to the total weight of the composition.

10. The composition according to claim 1, wherein the associative polyurethane is a copolymer comprising at least two hydrocarbon-based lipophilic chains containing from 6 to 30 carbon atoms, separated by a hydrophilic block.

11. The composition according to claim 10, wherein the hydrophilic block is an oxyethylene chain.

12. The composition according to claim 1, wherein the associative polyurethane is a triblock polymer.

13. The composition according to claim 1, wherein the associative polyurethane is water-soluble.

14. The composition according to claim 1, wherein the aqueous phase represents 10%-95% by weight relative to the total weight of the composition.

15. The composition according to claim 1, wherein the oily phase comprises at least one oil selected from the group consisting of silicone oils, fatty acid esters, and mixtures thereof.

16. The composition according to claim 1, wherein the amount of oil(s) is at least 5% by weight relative to the total weight of the composition.

17. The composition according to claim 1, furthermprising at least one emulsifier.

18. The composition according to claim 1, in the form of an oil-in-water emulsion.

19. The composition according to claim 18, further comprising at least one emulsifier selected from the group consisting of oxyalkylenated fatty acid esters of sorbitol.

20. The composition according to claim 1, wherein said composition is a dermatological or cosmetic composition.

21. The composition according to claim 1, wherein said composition is contained in container means.

22. A method, comprising applying the composition of claim 1 to a human keratin material.

23. The method according to claim 22, wherein said human keratin material is selected from the group consisting of skin, lips, eyelashes, eyebrows, nails and hair.

24. The method according to claim 23, wherein the keratin material is skin.
